(19) United States
(12) Patent Application Publication
(10) Pub. No.: US 2024/0173050 A1
(43) Pub. Date: May 30, 2024

(54) INSERTION AND ACCESS DEVICE FOR SURGICAL SYSTEM

(71) Applicant: Virtual Incision Corporation, Lincoln, NE (US)

(72) Inventors: Mark Reichenbach, Lincoln, NE (US); Shane Farritor, Lincoln, NE (US); David Matsuura, Solana Beach, CA (US); Jacob A. Moebius, San Diego, CA (US); Nelson M. Siu, Encinitas, CA (US)

(21) Appl. No.: 18/516,078

(22) Filed: Nov. 21, 2023

Related U.S. Application Data
(60) Provisional application No. 63/384,502, filed on Nov. 21, 2022.

(51) Int. Cl. A61B 17/34; A61B 17/02

(52) U.S. Cl. CPC A61B 17/3423; A61B 17/0218; A61B 17/3474; A61B 2017/3464

(57) ABSTRACT

Systems, devices, and/or methods relating to surgical procedures, and more specifically for providing access to an insufflated cavity of a patient and/or positioning surgical systems or devices into the cavity during surgical procedures. Access and insertion devices configured to couple with sealing devices, such as sealable sleeve devices and wound retractor devices. Access and insertion devices configured to provide fluidic sealing about a portion of a surgical device during surgical procedures.

### RELATED APPLICATIONS

This application claims priority to U.S. Provisional Patent Application No. 63/384,502, filed Nov. 21, 2022, the entire contents of which are incorporated herein by reference.

### TECHNICAL FIELD

The various embodiments herein relate to systems, devices, and/or methods relating to surgical procedures, and more specifically for accessing an insufflated cavity of a patient and/or positioning surgical systems or devices into such a cavity of a patient.

### BACKGROUND

Invasive surgical procedures are essential for addressing various medical conditions. When possible, minimally invasive procedures, such as laparoscopy, are preferred.

However, known minimally invasive technologies such as laparoscopy are limited in scope and complexity due in part to: (1) mobility restrictions resulting from using rigid tools inserted through access ports, and (2) limited visual feedback. Further, the technologies are also limited due to difficulties relating to maintaining access to the surgical cavity while also maintaining insufflation of the cavity. There is a need in the art for improved surgical methods, systems, and devices.

### SUMMARY

Discussed herein are various surgical access and insertion devices and methods. Some of the embodiments described herein build upon the concepts disclosed in U.S. Published Pat. No. 2021/0244439, the contents of which are hereby incorporated by reference in relevant part.

In some embodiments of this disclosure, a surgical insertion device comprises a flexible canister defining a lumen, a top cap coupled to a proximal end of the canister, and an incision port removably coupled to a distal end of the canister, the incision port operably coupled to a first fluidic sealing component configured to maintain a fluidic seal at the distal end of the canister. The canister is sized to receive a surgical device in the lumen of the canister. The top cap comprises a lumen defined in the top cap, and a flexible seal having an opening formed therein, wherein the lumen is configured to receive a device body of a surgical device slidably disposed through the opening formed in the flexible seal. The flexible seal of the top cap may be formed of a silicone material and/or may comprise a wavy or sinusoidal cross-section to enable the flexible seal to conform to an outer surface of the device body of the surgical device. The flexible seal can thereby comprise a second fluidic sealing component forming a fluidic seal about the device body of the surgical device at the proximal end of the canister while allowing for relative slidable movement between the device body and the flexible seal.

In some embodiments, the first fluidic sealing component of the incision port may comprise a sealable sleeve device or a wound retractor seal device. In some embodiments, the first fluidic sealing component is a wound retractor seal configured to couple to the incision port by being flexibly disposed within at least two inwardly-directed radial tabs formed at a distal portion of the incision port. In some embodiments, one or more of the inwardly-directed radial tabs may comprise a rotatable latch. In some embodiments, the first fluidic sealing component is a wound retractor seal configured to couple to the incision port by being flexibly disposed around an outer portion of a plurality of barbs or tabs formed at a distal portion of the incision port. In some embodiments, the plurality of barbs or tabs extend downwardly from a portion of the incision port and are configured to flex or bend radially inwardly to enable coupling of an upper ring portion of the wound retractor seal to an outer portion of the plurality of barbs/tabs.

In some embodiments, a surgical insertion device according to this disclosure may include an iris or aperture style seal at an upper portion of the incision port. For example, see: https://www.sciencedirect.com/science/article/pii/S0015028204000135, which describes a “Lap Disk,” an iris (or aperture or lens) style sealing mechanism and opening that is activated (e.g., opened or closed) via a rotational actuation mechanism. The iris seal mechanism may, for example, include a rotatable ratcheting closure function. In some embodiments, a surgical insertion device according to this disclosure may include an insufflation port fluidly coupled to the incision port; in some cases, the insufflation port may couple to the incision port below (e.g., distal to) the level of the iris seal and above (e.g., proximal to) the level of the wound retractor.

In some embodiments, an introducer for use in conjunction with a surgical access or insertion device is provided herein. For example, an introducer may comprise a pair of downward extending introducer legs configured to be positioned within an opening in a top cap seal, for example, and may be moved apart after being positioned within the opening in the top cap seal. In some embodiments of this disclosure, a pair of relatively long introducer legs may be flexibly coupled together at an upper portion and biased towards a spread-apart configuration; use thereof may involve urging the legs together (e.g., by a surgeon or other healthcare practitioner) prior to insertion into the top cap opening, for example. In some embodiments of this disclosure, a pair of introducer legs may comprise individual components, each with a tab or protrusion formed thereon such that the tab or protrusion is configured to engage in a slot or receptacle portion of a top ring of the insertion device to thereby hold the top cap opening in a more fully open position, for example, after the legs have been placed within the top cap opening. In yet other embodiments, a pair of introducer legs may be pivotably coupled together at a pivot joint with a handle portion associated with each introducer leg such that the legs and handle portion operate in a manner similar to that of a scissors. That is, following placement of the introducer legs within the top cap opening, the introducer legs may be moved apart (e.g., radially outward) towards a spread-apart configuration via movement of the handle portions towards each other (“squeezing” them together), for example.

In some embodiments, rather than using a wound retractor or sealable sleeve device coupled to the incision port of the surgical insertion device, a trocar-style alternative wound attachment may instead be employed at the incision site of the patient. The trocar-style alternative wound attachment may be configured to couple to a lower portion of the flexible canister, for example, to thereby form an alternative surgical insertion device according to certain embodiments of this disclosure.

In another embodiment of this disclosure, a surgical insertion device comprises a flexible canister defining a lumen, a top cap coupled to a proximal end of the canister, and an incision port removably coupled to a distal end of the canister, the incision port comprising a first fluidic sealing component configured to maintain a fluidic seal at the distal end of the canister. The canister is sized to receive a surgical device in the lumen. The top cap comprises at least one lumen defined in the top cap, wherein the at least one lumen is configured to receive a device body of a surgical device slidably disposed therethrough. The at least one lumen of the top cap may comprise a second fluidic sealing component shaped to conform to an outer surface of the device body, the second fluidic sealing component rotatably disposed within the top cap to enable rotation of the surgical device relative to the top cap while maintaining a fluidic seal about the device body of the surgical device at the proximal end of the canister.

In some embodiments of this disclosure, the flexible canister of the surgical insertion device has a conical shape. In some embodiments of this disclosure, the flexible canister of the surgical insertion device facilitates tilting of the surgical device disposed therethrough in any direction. In some embodiments of this disclosure, the distal end of the flexible canister is configured to be removably coupled to the incision port via an adapter clamp configured for single-hand operation. In some embodiments of this disclosure, the adapter clamp further comprises a pressure relief valve.

While multiple embodiments are disclosed, still other embodiments of the present disclosure will become apparent to those skilled in the art from the following detailed description and accompanying drawings, which show and describe illustrative embodiments. As will be realized, the devices and/or methods described herein are capable of modifications in various obvious aspects, all without departing from the nature and scope of the present disclosure. Accordingly, the drawings and detailed description are to be regarded as illustrative in nature and not restrictive.

### BRIEF DESCRIPTION OF DRAWINGS

FIG. 1 is a top perspective view of a system or apparatus for positioning a surgical system or device into a body cavity of a patient, according to some embodiments;

FIG. 2A is a bottom perspective view of an incision port portion of a system or apparatus for positioning a surgical system or device into a body cavity of a patient, according to some embodiments;

FIGS. 2B and 2C are bottom perspective views of an alternative incision port portion of a system or apparatus for positioning a surgical system or device into a body cavity of a patient, according to some embodiments;

FIG. 3A is a bottom perspective view of another alternative incision port portion of a system or apparatus for positioning a surgical system or device into a body cavity of a patient, according to some embodiments;

FIG. 3B is a partial, cross-sectional perspective view of the incision port portion of FIG. 3A;

FIG. 4A is a top perspective view of an incision port portion of a system or apparatus for positioning a surgical system or device into a body cavity of a patient, according to some embodiments;

FIG. 4B is a partial cross-sectional view showing an exemplary arrangement of components of the incision port portion of FIG. 4A;

FIG. 5A is top perspective view of an incision port portion of a system or apparatus for positioning a surgical system or device into a body cavity of a patient, showing aspects of a flexible seal arrangement according to some embodiments;

FIG. 5B is a side cross-sectional view of the incision port portion of FIG. 5A, showing additional aspects of the flexible seal arrangement according to some embodiments;

FIG. 5C is an enlarged, partial, side cross-sectional view of the incision port portion of FIGS. 5A and 5B, showing additional aspects of the flexible seal arrangement according to some embodiments;

FIGS. 5D and 5E are top perspective views of a system or apparatus for positioning a surgical system or device into a body cavity of a patient, showing views of the flexible seal arrangement in an open and a closed configuration, respectively, according to some embodiments of this disclosure;

FIG. 6A is a top perspective view of a system or apparatus for positioning a surgical system or device into a body cavity of a patient, showing views of the flexible seal arrangement in an open and a closed configuration, respectively, according to some embodiments of this disclosure;

FIG. 6B is a partial cross-sectional side view of the system or apparatus of FIG. 6A;

FIG. 7A is a top perspective view of a top cap portion and a top cap seal of an exemplary system or apparatus for positioning a surgical system or device into a body cavity of a patient, according to some embodiments of this disclosure;

FIG. 7B is a cross-sectional side view of the top cap and top cap seal portions of FIG. 7A;

FIGS. 8A and 8B are top perspective views showing an embodiment of an introducer for facilitating insertion of a surgical device through a top cap seal of a system or apparatus according to some embodiments of this disclosure;

FIGS. 9A and 9B are a top perspective view and a schematic cross-sectional view, respectively, showing an alternative embodiment of an introducer for facilitating insertion of a surgical device through a top cap seal of a system or apparatus according to some embodiments of this disclosure;

FIGS. 10A and 10B are top perspective views showing another alternative embodiment of an introducer for facilitating insertion of a surgical device through a top cap seal of a system or apparatus according to some embodiments of this disclosure;

FIG. 11A is a perspective view of an external pressurized system or apparatus for positioning a surgical system or device into a cavity of a patient, according to some alternate embodiments;

FIG. 11B is a cross-sectional view of an external pressurized system or apparatus, including an incision port comprising a sealable sleeve device, according to the embodiment of FIG. 11A;

FIGS. 12A and 12B are perspective views of the external pressurized system or apparatus of FIG. 11A, showing a surgical system or device being rotated from a first position to a second position within the external pressurized system or apparatus;

FIG. 13A is a top view of a top cap, according to one embodiment;

FIG. 13B is perspective view of the top cap of FIG. 13A;

FIG. 13C is a cross-sectional view of the top cap of FIG. 13A;

FIG. 14 is side view of the external pressurized system or apparatus of FIG. 11A, showing the surgical system or device tilted within the external pressurized system or apparatus, according to one embodiment;

FIG. 15A is a partially exploded perspective view of a canister and an adapter clamp for the external pressurized system or apparatus of FIG. 11A, according to one embodiment;

FIG. 15B is a partial side cross-sectional view of the canister and an adapter clamp for the external pressurized system or apparatus of FIG. 11A, according to one embodiment;

FIG. 15C is a cross-sectional view of the canister and top cap for the external pressurized system or apparatus of FIG. 11A, according to one embodiment;

FIG. 16A is a side view of the external pressurized system or apparatus of FIG. 11A, according to one embodiment;

FIG. 16B is an enlarged side view of an adapter clamp for the external pressurized system or apparatus of FIG. 16A having a pressure relief, according to one embodiment;

FIGS. 17A and 17B are cross-sectional and exploded side views, respectively, of an alternative wound attachment assembly for use with the external pressurized systems or apparatuses of this disclosure according to an alternative embodiment; and

FIGS. 17C, 17D, and 17E are cross-sectional, top perspective, and schematic side views, respectively, of an alternative wound attachment assembly for use with the external pressurized systems or apparatuses of this disclosure according to an alternative embodiment.

### DETAILED DESCRIPTION

The various embodiments described herein relate to systems, devices, and/or methods for accessing an insufflated cavity of a patient and/or positioning surgical systems or devices into such a cavity of a patient. This disclosure builds upon devices, methods, and systems described in U.S. Published Pat. 2021/0244439, the contents of which are hereby incorporated by reference in relevant part.

Certain embodiments provide for insertion of the surgical systems/devices into a body cavity of a patient while maintaining sufficient insufflation of the cavity. For example, some embodiments reduce the complexity of the access/insertion procedure and/or facilitate performing the steps required for the procedure. Other embodiments relate to devices that have minimal profiles, minimal size, or are generally minimal in function and appearance to enhance ease of handling and use.

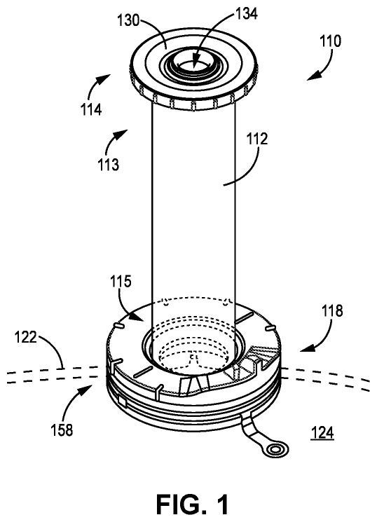

An exemplary embodiment of this disclosure relates to an external pressurized system or apparatus. For example, one implementation of an external pressurized system or apparatus 110 is depicted in FIG. 1. The apparatus 110 comprises a canister 112 with a top cap 114 coupled to a top portion 113 of the canister 112. In this embodiment, the device 110 includes an incision port 118 that is coupled to the canister 112 at a base portion 115 of the canister 112. The incision port 118 is configured to be positioned in an incision 158 formed in the skin 122 of the patient, thereby providing access to a body cavity 124 of the patient. As shown in FIG. 1, the top cap 114 of apparatus 110 may include a top cap seal portion 130 with a top cap lumen or opening 134 formed therein. Top cap lumen 134 may be configured to receive a portion of a surgical device (not shown in FIG. 1) to facilitate insertion of the surgical device into the body cavity 124 of the patient via the apparatus 110 and through the incision port 118, according to some embodiments.

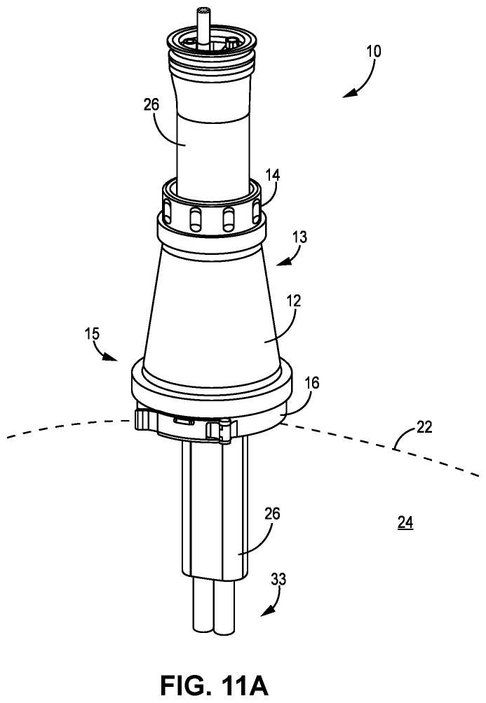

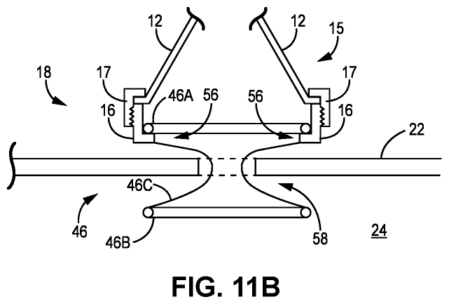

With continued reference to FIG. 1, the external pressurized device 110 may be used to enable a surgical device (e.g., a robotic surgical device having one or more robotic arms coupled thereto) to access and/or be positioned for use within a body cavity 124 of a patient, according to some embodiments. For example, it may be desirable to position a robotic surgical device (see, for example, FIG. 11A, which shows a robotic surgical device 26 having one or more end effectors 33 disposed at a distal end thereof, as depicted in use with an alternate embodiment of an access and insertion device 10 of this disclosure) within a body cavity 124 of a patient, such as the peritoneum for example, in order to perform a minimally invasive surgical procedure in the body cavity 124. In such a procedure, it is often desirable to insufflate the body cavity 124 with an insufflation gas such as carbon dioxide in order to create a working space within the body cavity 124. The insufflation gas may be provided to body cavity 124 at a predetermined pressure to provide the desired amount of working space for the surgical procedure, while being careful not to over-pressurize the body cavity 124. It may be desirable to maintain the insufflation pressure within a certain range; accordingly, it may be likewise desirable to selectively seal and/or open the access to the body cavity 124 via the incision port 118 in order to maintain the desired insufflation pressure.

As noted above, the incision port 118 may be placed in an incision 158 in a patient's skin 122 to create a seal in the area around the incision 158 that fluidly seals the patient's body cavity 124 from the ambient air outside the patient. For example, an incision 158 may be formed in the patient's skin 122 to provide access to the patient's body cavity 124, which, in some embodiments, can be the peritoneal cavity, but could be any known body cavity. Once the incision 158 has been formed, a fluidic sealing element, such as a wound retractor seal or sealable sleeve device (not shown in FIG. 1) may be positioned in the incision 158. An example of a commercially available wound retractor seal that may work well with the embodiment of device 110 described herein is the SURGISLEEVE™ Wound Protector distributed by Medtronic, Inc. Another example of a “standard” sealable sleeve device that may work well with certain embodiments described herein is a commercially-available retractor port, such as the DEXTRUS® Retractor available from Ethicon Endo-Surgery. The wound retractor or sealable sleeve device may be positioned through the incision 158, for example, by inserting a lower ring of the wound retractor seal through the incision such that the lower ring (or lower sleeve ring) is positioned within the patient's body cavity 124, and an upper ring (or upper sleeve ring) is positioned outside the patient, with a flexible sleeve portion extending through the incision 158. In some cases, the upper and/or lower sleeve rings of the wound retractor or sealable sleeve device are flexible rings that can be deformed, e.g., to facilitate insertion through the incision 158 and/or to facilitate coupling to the incision port 118.

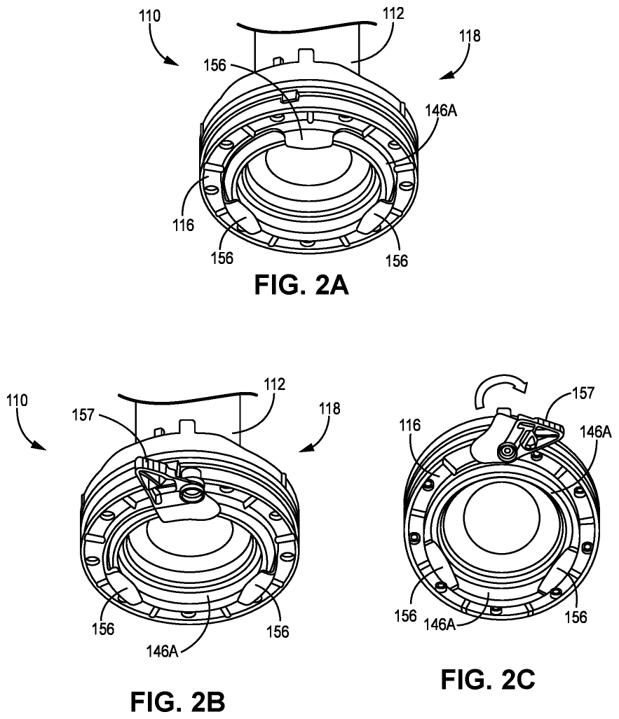

FIG. 2A shows a partial bottom perspective view of access device 110 according to some embodiments. In the embodiment depicted, a lower portion of the incision port 118 includes a number of ledges or shelf portions 156 disposed radially about the circumference of a lumen formed within the incision port 118. The embodiment of FIG. 2A shows three such ledges 156 disposed about the lumen of the incision port 118; however, one of ordinary skill in the art would readily recognize that more or fewer ledges 156 may be employed to achieve a similar effect. Each ledge 156 extends downwardly and radially inwardly (e.g., from a lower ring 116 of incision port 118) to form a “shelf” upon which an upper sleeve ring 146A of a wound retractor may be seated or coupled. (Note: Only the upper sleeve ring 146A of the wound retractor is shown in FIGS. 2A-2C for clarity.) For example, the upper sleeve ring 146A of the wound retractor may be flexed and/or bent as needed to seat the upper portion of the wound retractor in the ledges 156 and thereby couple the wound retractor to the incision port 118 of device 110.

FIGS. 2B and 2C show a variation of the embodiment depicted in FIG. 2A. In the embodiment depicted in FIGS. 2B and 2C, one or more of the ledges 156 is replaced by a pivotable latch 157 that can rotate out of the way (as shown in FIG. 2C) to facilitate placement of an upper ring 146A of a wound retractor in coupling engagement with an inner portion of the incision port 118, and then pivotable latch 157 may rotate back into position (as shown in FIG. 2B) to hold the upper ring 146A in place (e.g., in engagement with incision port 118). In some embodiments, the rotation of pivotable latch 157 is about a pivot point formed in the lower ring 116 of incision port 118. In some implementations, it may be desirable for the pivotable latch 157 to be biased toward one position or the other, for example, pivotable latch 157 may be spring-biased toward the engagement position of FIG. 2B.

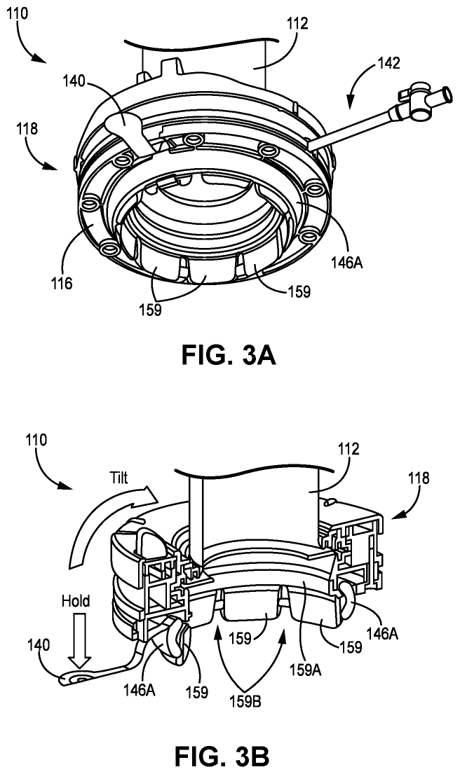

FIG. 3A shows a partial bottom perspective view of an access device 110 according to some embodiments. In the embodiment depicted, a lower portion of the incision port 118 includes a plurality of barbs or tab portions 159 disposed about the circumference of a lumen formed within the incision port 118, the plurality of barbs or tab portions 159 extending downwardly from a portion of the incision port 118. In some embodiments, the plurality of tabs 159 may extend downwardly from a common ring 159A, as shown in FIG. 3B, and could either be integrally formed with common ring 159A, or individually coupled to common ring 159A. As indicated in FIG. 3B, a series of gaps or spaces 159B may be formed between adjacent tabs 159. The spacing of adjacent tabs 159 in this manner may provide some level of radial flexibility such that the upper ring 146A of a wound retractor may be coupled about the outer surfaces of the plurality of tabs 159 by flexing or bending one or more of the tabs 159 radially inward to thereby position the upper ring 146A around the tabs 159. Once placed, the flexed tabs 159 are allowed to flex back (e.g., radially outward) to their normal/rest position such that an inner surface of the upper ring 146A is seated and/or coupled with an outer surface of the plurality of tabs 159, substantially as shown in FIG. 3B.

Also shown in FIGS. 3A and 3B is a removal tab 140 configured to facilitate removal of the wound retractor from the incision port 118. Removal tab 140 accomplishes this by pulling outwardly on the upper ring 146A of the wound retractor while simultaneously pushing inwardly on one or more of the tabs 159. In some embodiments, removal of the wound retractor from the incision port 118 can be further facilitated by tilting and/or pulling the device 110 in a direction generally opposite the direction in which the removal tab is pushed and held, for example. FIG. 3A also shows an insufflation port 142 that may be included as part of the access device 110; insufflation port 142 will be described in more detail below.

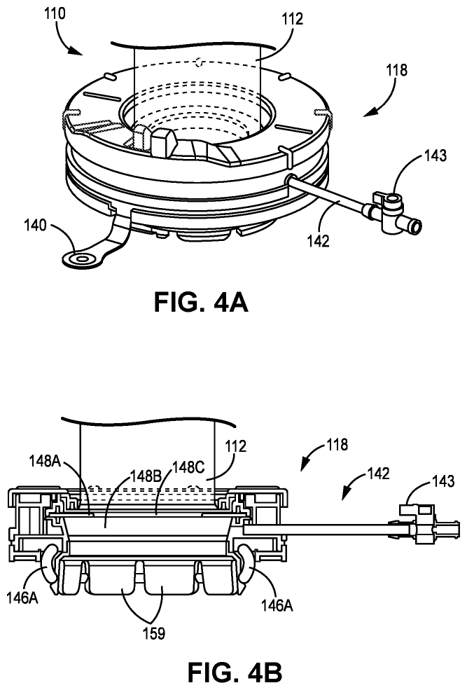

FIG. 4A is an enlarged perspective view of a portion of an exemplary access device 110 having an insufflation port 142 coupled to the incision port 118 of the device 110. FIG. 4B provides a cross-sectional side view of the incision port 118, showing the arrangement and/or placement of the insufflation port 142 relative to the various components of the incision port 118. FIGS. 4A and 4B both show a valve 143 disposed on the insufflation port 142 to control the flow of air from an air source, for example. In some cases, the valve 143 is a two-way stopcock 143 coupled to an outer end of insufflation port 142. As detailed in FIG. 4B, insufflation port 142 is configured to supply air or gas from a supply source (not shown) to an internal portion of incision port 118. As shown, insufflation port 142 is configured to fluidly couple an air supply (or other gas source) at a level in the incision port 118 above the wound retractor or sealable sleeve device. It is also noted that the insufflation port 142 is configured to fluidly couple an air supply (or other gas source) at a level in the incision port 118 that is below the level of an “iris” sealing mechanism disposed in the incision port 118. As seen in FIG. 4B, the insufflation port 142 fluidly couples to a lumen or cavity within the incision port 118 that is beneath a selectively sealable iris opening 148C. The iris sealing mechanism comprises a lower iris ring 148B and an upper iris ring 148A; operation of the iris sealing mechanism to selectively open and close the iris opening 148C is described in more detail below.

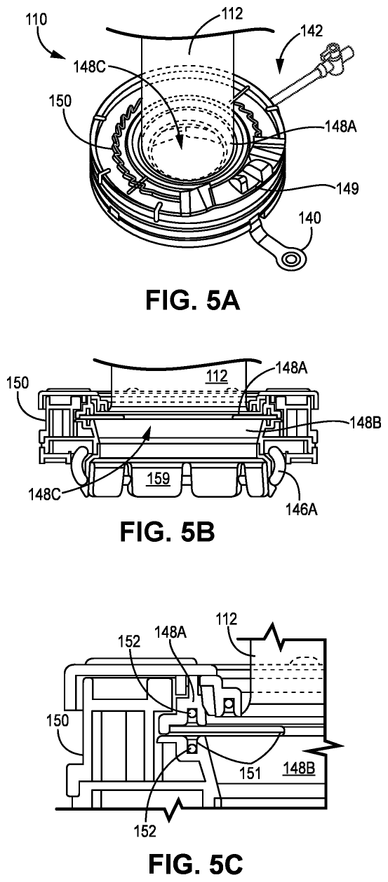

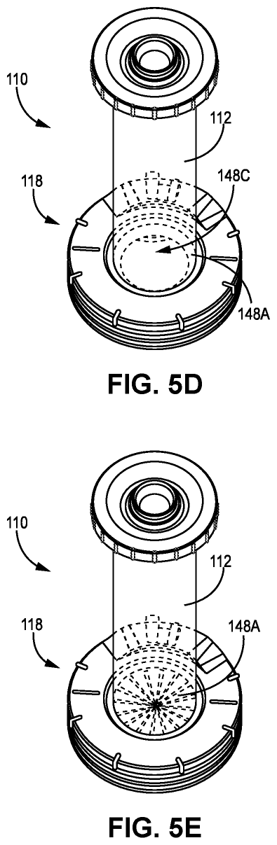

FIGS. 5A-5C illustrate an exemplary arrangement of various components of the incision port 118, including components that may form the iris sealing mechanism referred to above. For example, FIG. 5A is a partial top perspective view of an access device 110 with certain portions depicted in transparent outline form to show a ratchet ring 150 disposed within incision port 118. The iris sealing mechanism operates to selectively open and close the iris opening 148C via rotation of upper iris ring 148A and lower iris ring 148B relative to one another. The ratchet mechanism of ratchet ring 150 may operate to help prevent inadvertent opening of the iris opening 148C; that is, rotation of the upper iris ring 148A in one direction relative to the lower iris ring 148B (e.g., clockwise) operates to close the iris opening 148C, while rotation in the opposite direction (e.g., counterclockwise), if not prevented by the ratchet mechanism of ratchet ring 150, would operate to open iris opening 148C. A slider mechanism 149 may be employed to provides a release to “override” the ratchet mechanism and thereby enable opening of the iris opening 148C upon concurrent actuation of the slider mechanism 149 and counterclockwise rotation of the upper iris ring 148A and lower iris ring 148B relative to one another, for example.

FIG. 5B is a cross-sectional side view of the elements described above with respect to the iris seal mechanism. FIG. 5C is an enlarged partial cross-sectional side view showing additional details of the arrangement of components associated with the iris seal mechanism. For example, in FIG. 5C, a flexible sealing material 151 is shown disposed on a portion of upper iris ring 148A such that it opens and closes with operation of the iris seal mechanism and forms a fluidic seal when closed. The flexible sealing material 151 is shown to extend around an upper and lower surface of upper iris ring 148 and is held in place via a pressed-in bead 152 mechanically engaged in channels formed in both the upper and lower iris rings 148A and 148B as shown.

FIGS. 5D and 5E are top perspective views of an access device 110 having an iris sealing mechanism, showing the iris opening 148C and upper iris ring 148A as they would appear when the iris sealing mechanism is in an open position (FIG. 5D), and showing the upper iris ring 148A as it might appear when the iris sealing mechanism is in a closed position (FIG. 5E).

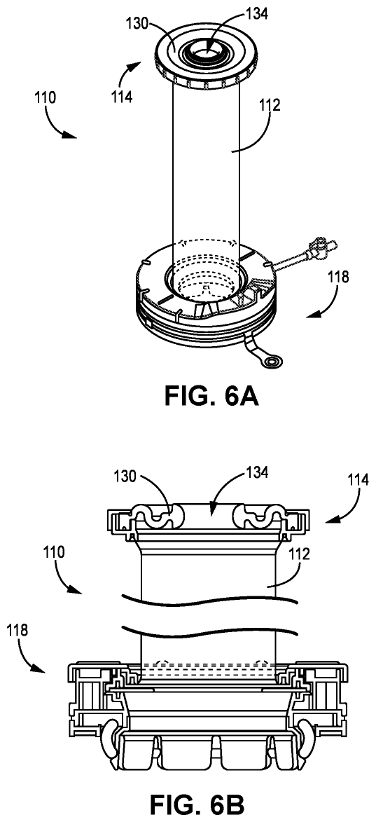

FIG. 6A is a top perspective view of an access device 110 according to some embodiments of this disclosure, the access device 110 comprising a top cap 114, a canister 112, and an incision port 118. The top cap 114 is coupled to an upper portion of the canister 112 and includes a top cap seal portion 130 disposed across an upper surface of the top cap 114. The top cap seal portion 130 has a top cap lumen or opening 134 formed therein. As described previously, the top cap lumen 134 may be configured to receive a portion of a surgical device (not shown in FIG. 6A) to facilitate insertion of the surgical device into a body cavity of a patient via the apparatus 110, the surgical device configured to pass through the top cap 114, through the canister 112, and through the incision port 118, to gain access to a body cavity during a surgical procedure, for example. The top cap seal portion 130 and the top cap opening 134 are formed to provide a fluidic seal about a portion of the surgical device while slidably moving the surgical device through the top cap opening and/or moving the surgical device relative to the top cap seal portion 130.

FIG. 6B is a cross-sectional side view of the device 110 of FIG. 6A showing details for an exemplary top cap seal portion 130 and opening 134 that may be used with device 110 according to some embodiments. In some embodiments, canister 112 may be formed of a TPU film stock, for example. In some embodiments, canister 112 may have a relatively long length or height, which may be desirable to enable some amount of rotation or twisting of canister 112 during movement and positioning of a surgical device inserted therethrough. In FIG. 6B, it is also noted that the wall material of canister 112 may be held in place at either or both the upper and lower portions via a pressed-in bead arrangement, for example, formed in a lower portion of the top cap 114 and/or in a ring of the incision port 118.

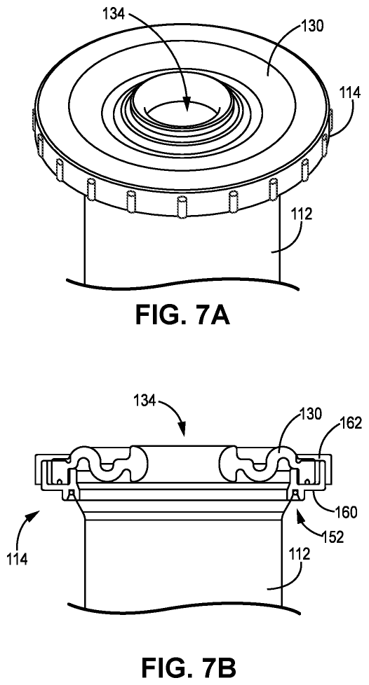

FIGS. 7A and 7B are enlarged views (top perspective view and cross-sectional side view, respectively) of the top cap 114 of device 110 showing additional structural details that may be associated with the top cap seal 130 and/or opening 134. FIG. 7B, for example, shows a cross-section of top cap seal 130 having a wavy or sinusoidal shape between an outer perimeter of seal 130 and the opening 134. The seal 130 may be formed of silicone; in some embodiments, the silicone will maintain a seal about a surgical device body even while the device body translates and/or rotates relative to the seal 130 and the seal opening 134. In some embodiments, the silicone of seal 130 may comprise a molded silicone having a 00-50 durometer. In some further embodiments, a parylene-C coating on the silicone of seal 130 may provide a lubricious coating such that additional lubricant may not be needed to retain a slidable relationship between the seal 130 and a portion of a surgical device disposed therethrough. The wavy or sinusoidal cross-sectional shape and/or the silicone material may contribute to the ability of the seal 130 to maintain a fluidic seal during such translational and/or rotational motion of the surgical device relative to the seal 130, and also doing so even when the surgical device does not have a matching round or circular cross-sectional shape. In some cases, the outer surface of the surgical device may, for example, comprise a D-shaped cross-section, yet the generally circular shape of opening 134 will compress and conform around the surgical device body to maintain a fluidic seal according to some embodiments of this disclosure.

FIG. 7B also shows a lower top cap ring 160 being shaped (e.g., recessed) to receive an outer portion of top cap seal 130 disposed therein. An upper top cap ring 162 is disposed as shown in FIG. 7B to retain the positioning of top cap seal 130 across top cap 114. Additionally, in some embodiments, the lower top cap ring 160 may have a lower recess formed in a lower portion thereof to facilitate placement and retention of a top portion of canister 112 via a pressed-in bead 152 arrangement generally as shown in FIG. 7B.

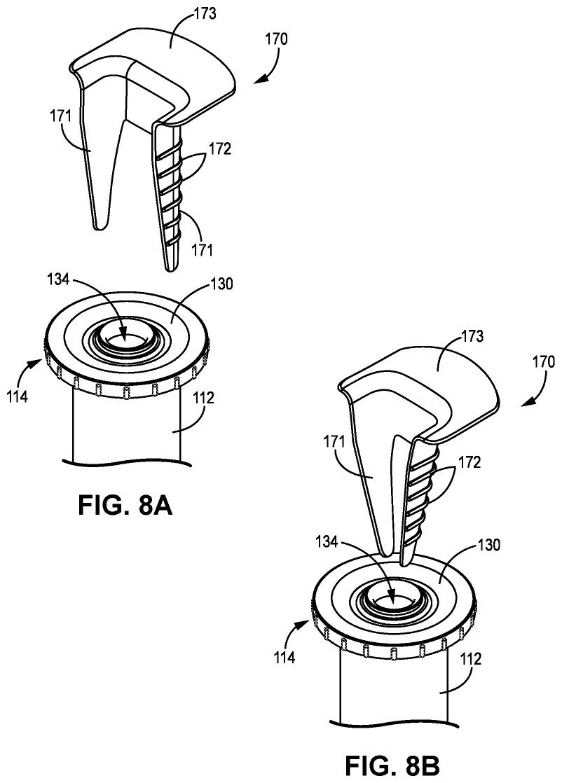

One or more introducer devices may be used in conjunction with the access and insertion device 110 of this disclosure in order to facilitate placement of a surgical device into a slidable, fluidically sealed engagement with the top cap seal 130 of top cap 114 of device 110. One example of an introducer for this purpose is shown in FIGS. 8A and 8B. Introducer 170 is shown in FIG. 8A in its relaxed or expanded configuration having a pair of legs 171 extending downwardly from a generally horizontal or flat top portion 173. FIG. 8B shows the introducer 170 as it would be prepared for use to hold open the opening 134 of seal 130. A user, for example, may “pinch” and hold the pair of legs 171 together while pushing them downwardly into opening 134. When positioned sufficiently within opening 134, the user may release the legs 171, allowing them to expand outwardly to their normal configuration, thereby holding open the opening 134 to facilitate placement and/or positioning of a portion of a surgical device therethrough. In some embodiments, a series of textures or ribs 172 may be disposed on an outer surface of the legs 171 to help keep the introducer from slipping upward and/or out of the opening 134 during use. Once the surgical device has been placed appropriately within opening 134 of seal 130, the introducer 170 may be removed from the top cap opening 134 by pulling upward and/or outward on the flat top portion 173 of introducer 170.

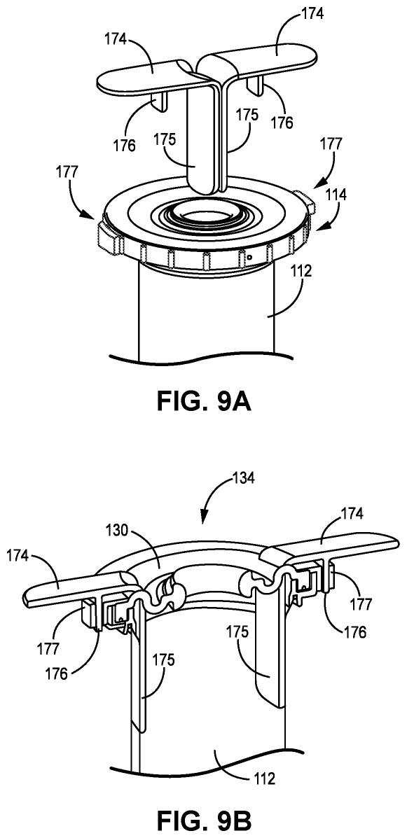

FIGS. 9A and 9B illustrate the design and use of another introducer embodiment that may be employed with device 110. A pair of introducers 174 may be used as shown in FIGS. 9A and 9B to hold open the opening 134 for surgical device insertion. Each introducer 174 has a downwardly extending leg 175 for placement in opening 134, and a downwardly extending tab 176 configured to mate with and/or seat within a slot or notch 177 formed in an outer portion of top cap 114 when introducers 174 are pulled apart sufficiently, as shown in FIG. 9B. Once the surgical device has been placed appropriately within opening 134 of seal 130, the introducers 174 may be removed by pulling upward and/or outward on the top portions of introducer 174.

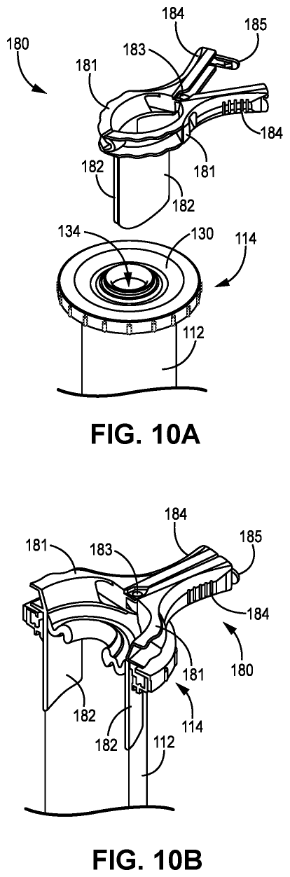

FIGS. 10A and 10B show another alternative introducer embodiment that may be employed with device 110 for positioning a surgical device in a sealing arrangement with a top cap 114 of device 110. As shown in FIG. 10A, a scissors-type introducer 180 may comprise two similar halves 181 pivotably coupled to each other at a pivot joint 183. Each half 181 comprises a leg portion 182 and a handle portion 184. In some embodiments, operation of the two handle portions 184 (e.g., single-handed operation) to bring the handle portions 184 together causes the corresponding leg portions 182 to spread apart from each other. In use, the legs 182 are positioned together as depicted in FIG. 10A, positioned downwardly into opening 134, then the handles 184 are actuated to move the legs 182 outwardly apart to thereby open the opening 134 to a more fully position suitable for placing a surgical device into and/or through the opening 134. In some embodiments, one or both handles may comprise a latch mechanism 185 to facilitate releasable latching or engagement of the handle portions 184 together to keep the opening 134 open during positioning of the surgical device therein. Once the surgical device has been placed appropriately within opening 134 of seal 130, the introducer 180 may be released and removed by disengaging the latching mechanism 185 (if used), moving the handle portions 184 apart from each other, and then pulling upward and/or outward on the introducer 180 to remove the legs from the opening 134.

An alternate embodiment of this disclosure relates to an external pressurized system or apparatus. For example, one implementation of an external pressurized system or apparatus 10 is depicted in FIGS. 11A and 11B. The apparatus 10 has a canister 12 with a top cap 14 coupled to a top portion 13 of the canister 12. In this embodiment, the device 10 includes an incision port 18 that is coupled to the canister 12 at a base portion 15 of the canister 12. The incision port 18 is positioned in an incision 58 formed in the skin 22 of the patient, thereby providing access to a body cavity 24 of the patient. As shown in FIGS. 11A and 11B, the apparatus 10 may be configured to receive a surgical device 26 such that the device 26 can be inserted into the patient cavity 24 through the apparatus 10 and the port 18.

With continued reference to FIG. 11A, the external pressurized device 10 as shown might be used to enable a surgical device 26 to access and/or be positioned for use within a body cavity 24 of a patient, according to some embodiments. For example, it may be desirable to position a robotic surgical device 26, for example having one or more end effectors 33 disposed at a distal end thereof, within a body cavity 24 of a patient, such as the peritoneum for example, in order to perform a minimally invasive surgical procedure in the body cavity 24. In such a procedure, it is often desirable to insufflate the body cavity 24 with an insufflation gas such as carbon dioxide in order to create a working space within the body cavity 24. The insufflation gas may be provided to body cavity 24 at a predetermined pressure to provide the desired amount of working space for the surgical procedure, while being careful not to over-pressurize the body cavity 24. It may be desirable to maintain the insufflation pressure within a certain range; accordingly, it may be likewise desirable to selectively seal and/or open the access to the body cavity 24 via the port 18 in order to maintain the desired insufflation pressure.

As noted above, the incision port 18 may be placed in the incision 58 to create a seal in the area around the incision 58 that fluidly seals the patient's body cavity 24 from the ambient air outside the patient. For example, an incision 58 may be formed in the patient's skin 22 to provide access to the patient's body cavity 24, which, in some embodiments, can be the peritoneal cavity, but could be any known cavity. Once the incision 58 has been made, a sealable sleeve device 46 (described below with reference to FIG. 11B) may be positioned in the incision 58. An example of a “standard” sealable sleeve device 46 is a commercially-available retractor port, such as the DEXTRUS® Retractor available from Ethicon Endo-Surgery. The sealable sleeve device 46 may be positioned through incision 58, for example, by inserting a lower sleeve ring 46B through the incision 58 such that the lower sleeve ring 46B is positioned within the patient body cavity 24, and the upper sleeve ring 46A is positioned outside the patient, with the flexible sleeve 46C extending through the incision 58. According to one embodiment, the lower sleeve ring 46B of the sealable sleeve device 46 is a flexible ring 46B that can be deformed such that it can be inserted through the incision 58.

In some embodiments, an adapter clamp 16 may be used to facilitate a relatively simple and/or quick coupling/connection between the base portion 15 of the canister 12 and the incision port 18. FIG. 11B is a cross-sectional view showing exemplary elements of the incision port 18, according to some embodiments. For example, FIG. 11B shows sealable sleeve device 46 positioned within incision 58 and engaged with adapter clamp 16 according to one embodiment. The upper sleeve ring 46A is shown engaging with one or more inwardly-directed protrusions 56 formed on an inner portion of adapter clamp 16. In some embodiments, there may be two or, preferably three or more such protrusions or “ledges” 56 on which the upper sleeve ring 46A may rest and/or engage to couple the canister 12 to the incision port 18, according to some embodiments. In some alternate embodiments, a continuous inwardly-directed ledge or shelf 56 (rather than a number of discrete protrusions) may be formed on an inner portion of adapter clamp 16 on which the upper sleeve ring 46A may engage. FIG. 11B further shows lower sleeve ring 46B positioned through incision 58 and within body cavity 24, with flexible sleeve 46C coupling upper and lower sleeve rings 46A and 46B together through the incision 58. Rotation of upper and lower sleeve rings 46A and 46B relative to each other may cause flexible sleeve 46C to “bunch” and to seal off the incision 58, and/or to urge upper and lower sleeve rings 46A and 46B towards each other, thereby enhancing the sealing engagement of clamp 16 towards the surface of the patient's skin 22.

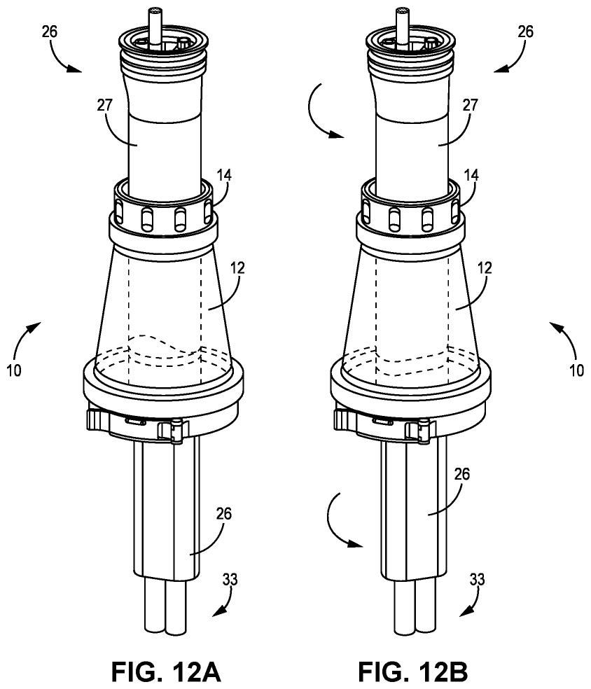

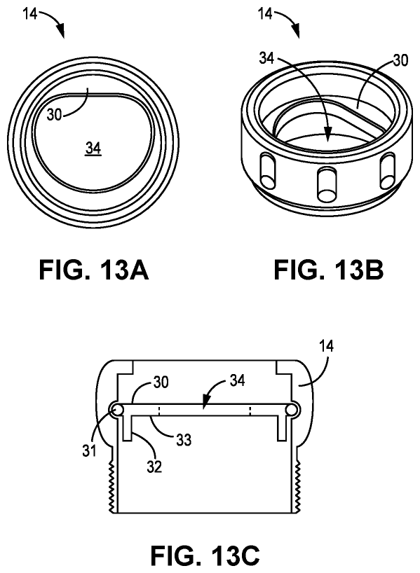

As shown in FIGS. 12A and 12B, in some embodiments of this disclosure, a surgical device 26 is configured to be disposed through the external pressurized device 10 (e.g., through a lumen formed in the top cap 14, through a length of canister 12, and extending through the incision 58 in the patient and into the body cavity 24). In some embodiments, the external pressurized device 10 comprises a rotatable sealing component (such as the rotatable component 30 depicted in FIGS. 13A and 13B) disposed within top cap 14 that enables surgical device 26 to rotate within and with respect to top cap 14 and/or the canister 12. For example, the surgical device 26 in FIG. 12A may be rotated counterclockwise approximately 90 degrees (as shown in FIG. 12B) in order to cause the same amount of rotation or change in orientation at a distal end of the surgical device 26, e.g., at the end effectors 33 disposed in the body cavity 24.

FIGS. 13A and 13B show an exemplary top cap 14 with a sealing component (or “rotatable seal”) 30 rotatably disposed within an inner portion of top cap 14. For example, sealing component 30 may have a generally circular outer shape that complements and/or mates with a generally circular inner surface of top cap 14. Sealing component 30 may be rotatably disposed within top cap 14 via a ball bearing design, as is known in the art. Alternatively, the rotatable seal 30 can be rotatably coupled to the top cap 14 via any known mechanism, device, or method. Sealing component 30 has a generally flat upper surface with a shaped lumen 34 disposed therein. In some embodiments, for example, the shaped lumen 34 may have a shape that conforms to an outer surface of a portion of the surgical device 26 (such as the device body 27 of a robotic surgical device 26, according to some embodiments) so as to form a seal around the aforementioned portion of the surgical device 26. More specifically, the shaped lumen 34 is substantially “D” shaped as best shown in FIG. 13A (and discussed in further detail below) such that the lumen 34 matches the outer cross-sectional circumferential shape of the body 27 of the surgical device 26 that can be positioned therethrough, thereby establishing a fluidic seal between the device body 27 and the seal 30.

Referring now to FIG. 13C, a side cross-sectional view of an exemplary top cap 14 is shown with sealing component 30 rotatably disposed therewithin via a ball bearing arrangement 31 formed in an inner surface of top cap 14. In the particular embodiment shown, sealing component 30 may comprise a substantially annular vertically extending portion 32 and a substantially flat or horizontal portion 33, with the shaped lumen 34 formed in the horizontal portion 33. In the exemplary embodiment shown in FIG. 13C, the horizontal portion 33 is disposed at an upper end of vertically extending portion 32; however, this orientation could be modified; that is, the horizontal portion 33 could be disposed at a lower end, or in the middle portion, of the vertically extending portion 32, with similar effect.

The sealing component 30 is configured to rotate in conjunction with rotation of the surgical device 26 so as to maintain a conforming seal around the outer surface of surgical device 26. In certain embodiments, this is due to the complementary shapes of the lumen 34 and the outer surface of a portion of surgical device 26. For example, in FIGS. 13A and 13B, lumen 34 is a generally “D”-shaped opening in sealing component 30 corresponding to a generally “D”-shaped outer surface of the device body 27 of surgical device 26. Of course, other appropriate complementary shapes could be chosen based on particular surgical device 26 being used. Thus, in use, when the surgical device 26 is rotated around the longitudinal axis of the device body 27, the rotatable seal 30 rotates along with the body 27.

In some embodiments, the seal formed by shaped lumen 34 around surgical device 26 may allow for some amount of slidable relative displacement of the device 26 in relation to the seal 30. For example, surgical device 26 may be able to slide vertically relative to the shaped lumen 34 to enable positioning of surgical device 26 within body cavity 24, while maintaining the seal formed around surgical device 26 by the sealing component 30. In some embodiments, the sealing component 30 may further comprise an actuation feature whereby a user may selectively engage and/or disengage the seal. For example, in some embodiments, an actuator (e.g., a button or latch, not shown) may be disposed on or near the top cap 14 to enable one-handed operation (e.g., engagement and/or disengagement) of the seal by a user. Such an actuator may, for example, function by temporarily releasing a circumferentially- or radially-directed tension in the sealing component 30 that exists during normal operation

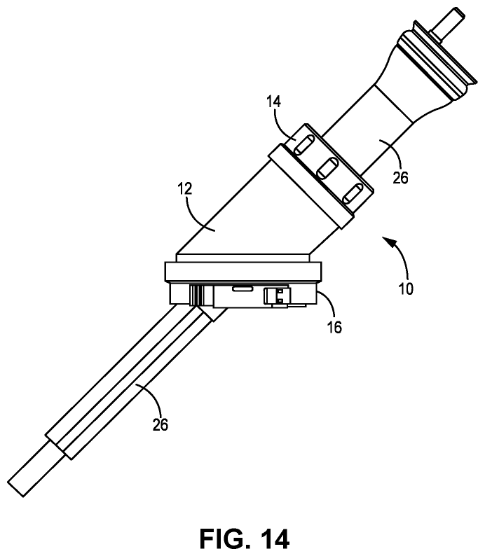

In some embodiments, the canister 12 of the external pressurized device 10 may be formed of a flexible material, which may thereby facilitate positioning and or manipulation of the surgical device 26 with respect to the body cavity 24 of the patient, as shown in the exemplary embodiment depicted in FIG. 14. For example, the canister 12 may be formed of flexible materials such as polyethylene plastic, latex, nylon, or silicone rubber. As such, the canister 12 may be manipulated and configurable with respect to the shape of the canister 12 and may further be configured to be compressed longitudinally such that the height of the canister 12 can be reduced during insertion of a robotic surgical device 26 into a patient's cavity 24.

The embodiment in FIG. 14 shows a flexible canister 12 that is generally conical in shape. According to one embodiment, during compression, the conical canister 12 may be configured to collapse upon itself or compress or otherwise allow the top cap 14 to be moved toward the incision port 18. In some embodiments, the flexibility of the material chosen for canister 12, and/or the length or height of canister 12, may enable a certain amount of tilting or bending of the canister 12 in various directions to facilitate positioning and use of the surgical device 26 disposed through canister 12. For example, in some embodiments, canister 12 may be generally conical in shape and may be formed to enable a surgical device 26 disposed therethrough to be bent or angled away from a generally vertical orientation by as much as 45 degrees or more in any direction. This may facilitate greater positioning flexibility of the surgical device 26 within the body cavity 24 of the patient, as suggested by the embodiment shown in FIG. 14.

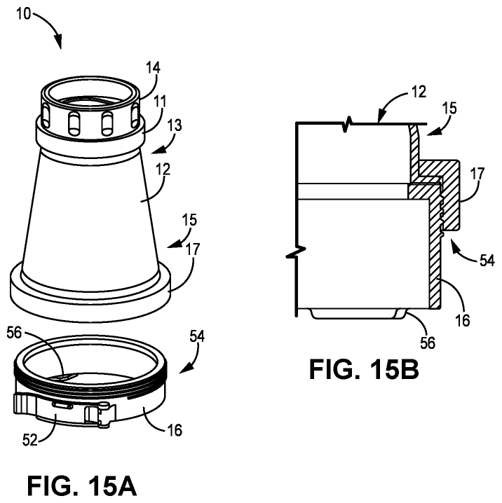

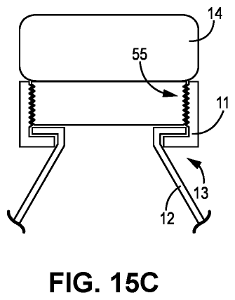

FIGS. 15A and 15B illustrate embodiments of an external pressurized device 10 comprising an adapter clamp 16 configured to facilitate quick and/or easy connection/coupling between a base portion 15 of a canister 12 and an incision port, such as the incision port 18 of FIG. 11B shown disposed in an incision 58 in a patient's skin 22. As noted above, incision port 18 may comprise a sealable sleeve device or a retractor port, such as the commercially available DEXTRUS® retractor port, for example.

As noted above, adapter clamp 16 may comprise one or more inwardly-directed protrusions 56 formed on an inner portion of adapter clamp 16, according to some embodiments. Alternately, adapter clamp 16 may comprise a substantially continuous inwardly-directed ledge or shelf 56 formed on an inner portion of adapter clamp 16, according to some embodiments. Protrusions or shelf 56 may form an engagement surface upon which a portion of a sealable sleeve device 46 (e.g., an upper sleeve ring 46A) is configured to seat and/or engage. In the embodiment shown in FIG. 15A, clamp 16 may further comprise a tightening latch 52 to facilitate forming a compressive engagement about an upper sleeve ring 46A of a sealable sleeve device 46, according to some embodiments.

With further reference to FIGS. 15A and 15B, additional details of the adapter clamp 16 are shown. For example, in FIG. 15A, threads 54 are formed on an outer, upper portion of clamp 16 to facilitate coupling to the canister 12 of device 10. FIG. 15B shows an enlarged cross-sectional view of the coupling between clamp 16 and the base portion 15 of canister 12. For example, threads 54 of clamp 16 may engage with corresponding threads of a lower sealing ring 17, which together are configured to compress or “sandwich” an outward extension of base portion 15 of canister 12 between an upper portion of clamp 16 and an inner portion of lower sealing ring 17 in a secure engagement as shown in FIG. 15B.

FIG. 15C is a cross-sectional side view of an exemplary coupling between top cap 14 and a top portion 13 of canister 12, showing a similar arrangement as that described above with respect to clamp 16. For example, complementary threads 55 formed on an outer lower surface of top cap 14 and on an inner surface of an upper sealing ring 11 may be used to tighten and thereby compress or “sandwich” an outward extension of top portion 13 of canister 12 between lower portion of top cap 14 and an inner lower portion of upper sealing ring 11 in a secure engagement as shown in FIG. 15C.

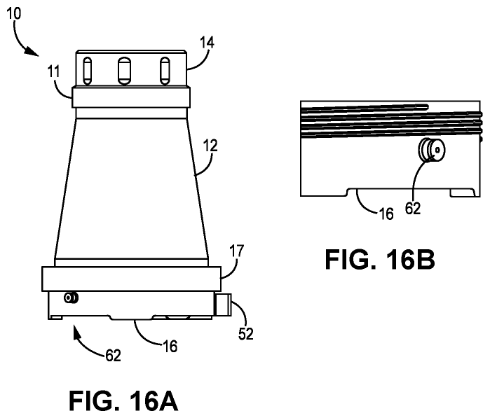

In some embodiments, adapter clamp 16 may further comprise a pressure relief valve 62, as shown in the embodiment depicted in FIGS. 16A and 16B. Pressure relief valve 62 may be configured to relieve pressure that may build up inside canister 12 and/or body cavity 24, for example. In some embodiments, the pressure relief valve 62 may be enabled or actuated to relieve pressure created during advancement (e.g., downward movement) of a surgical device 26 into the external pressurized device 10 and into body cavity 24. During such advancement of surgical device 26, canister 12 of device 10 may compress and or collapse somewhat upon itself, resulting in a reduced volume and a concurrent increase in pressure within the canister 12 and or the body cavity 24. This undesirable increase in pressure (which can be harmful to the patient) may be avoided by activation of pressure relief valve 62 to release the excess pressure upon reaching a certain predetermined pressure relief set point, for example. In one embodiment, the pressure relief valve 62 may comprise an elastomeric valve that deforms to relieve pressure when it reaches and/or exceeds a certain amount. Other forms of pressure relief valves 62 may be utilized as deemed appropriate by those of ordinary skill in the art.

In use, according to one embodiment, an external pressurized device according to any embodiment herein can be positioned for a surgical procedure in the following manner. First, in some implementations, the incision port (such as port 18) is first positioned in the incision to establish a fluidic seal between the patient cavity and the external, ambient air. In one embodiment, the insertion device 10 is coupled to the incision port 18 prior to positioning the port 18 in the incision. Alternatively, the port 18 is first positioned in the incision, and then the insertion device 10 is coupled to the port 18. Regardless, once the incision port 18 and insertion device 10 are positioned, the patient's cavity can then be insufflated. In certain implementations, due to the fluidic communication between the cavity and the interior of the insertion device 10 that can be created by the incision port 18, the entire interior of the insertion device 10 will be under the same pressure as the cavity.

In accordance with one implementation, once the insertion device 10 is positioned correctly, the process of inserting the robotic device 26 into the patient's insufflated cavity 24 can take place in the following manner. Initially, the robotic device 26 begins with both arms parallel and vertical to the incision, as best shown in FIGS. 12A and 12B. Then, the robot 26 is lowered through the lumen 34 in the top cap 14 and through an opening created by the incision port 18. In accordance with one embodiment, as the robotic device 26 is lowered such that the arms enter the patient cavity, the flexible canister 12 of insertion device 10 can shrink in height by allowing portions of the flexible material of the canister 12 to “crumple” or begin forming folds such that the top cap 14 moves closer to the incision port 18.

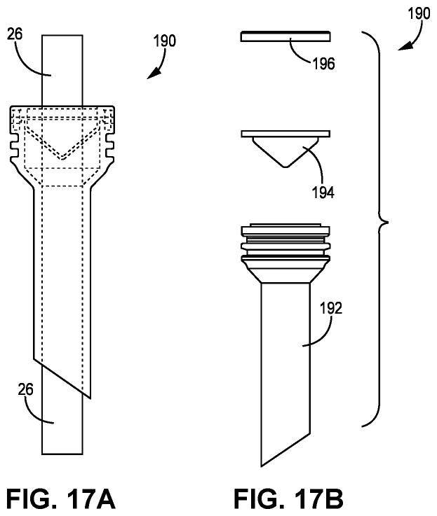

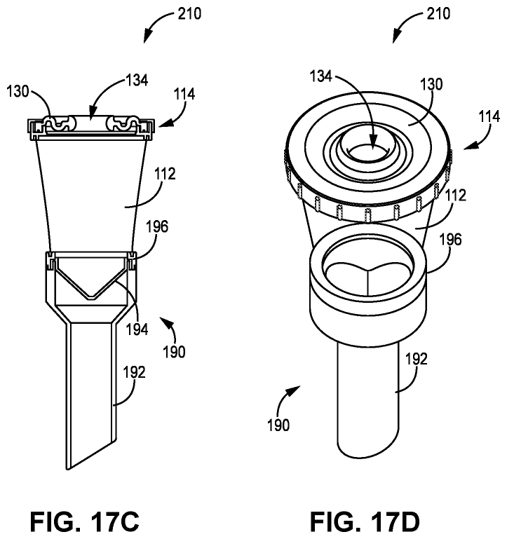

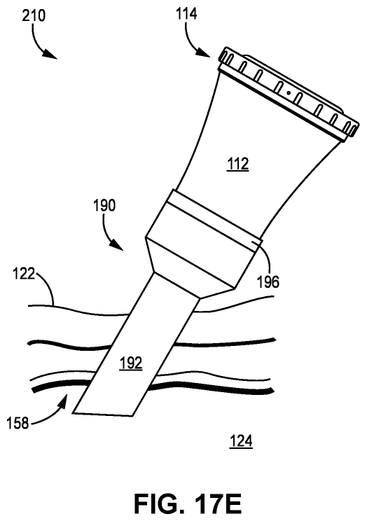

An alternative wound attachment assembly is depicted in FIGS. 17A and 17B. For example, FIG. 17A is a cross-sectional side view of an alternative wound attachment assembly 190 that may be used with any of the above-described external pressurized access or insertion devices 110 or 10 to facilitate access and/or insertion of a surgical and/or robotic device 26 into a body cavity 24 or 124 of a patient. FIG. 17B provides an exploded side view of the alternative wound attachment assembly 190 of FIG. 17A. The device 190 depicted in FIGS. 17A and 17B would function in place of the incision port and/or wound retractor devices discussed above to provide a seal at the incision of the patient. FIG. 17B shows components of this embodiment, which may include a trocar-style access device 192, a valve/seal device 194 (e.g., a duckbill or comparable style valve or seal), and an attachment ring 196 for engaging with a base portion of an access and insertion device 10/110 of this disclosure, for example. Notably, this embodiment does not involve the use of a sealable sleeve device or wound retractor seal as part of the incision port.

FIG. 17C is a cross-sectional side view of the alternative wound attachment assembly 190 of FIG. 17A as it might be used in conjunction with a canister 112 and top cap 114 to form an alternative access and/or insertion device 210. As shown, attachment ring 196 of wound attachment assembly 190 couples to a lower portion of canister 112. The remaining portions of canister 112 and top cap 114 are substantially as described above with reference to access device 110. FIG. 17D provides a top perspective view of the alternative access and/or insertion device 210, while FIG. 17E is a schematic side view of the alternative access and/or insertion device 210 showing its placement through an incision 158 formed in the skin 122 to enable access and insertion of a surgical device into a body cavity 124 of a patient in conjunction with performing a surgical procedure therein.

It is understood with respect to all of the various embodiments described herein that the medical devices being inserted into the patient are any known medical or surgical devices for performing procedures within a cavity of a patient. In certain embodiments, it is understood that the medical devices are robotic surgical devices having one or two arms. In various alternatives, the robotic surgical devices or systems can have or use three or more arms. In further alternatives, the devices (or additional devices) can be cameras or camera systems. Yet other alternatives, include the use of “helper” tools that can be inserted along with one or more medical devices or robotic devices.

Although the present invention has been described with reference to preferred embodiments, persons skilled in the art will recognize that changes may be made in form and detail without departing from the scope of this disclosure.

1. A surgical insertion device comprising:
(a) a flexible canister defining a canister lumen, the flexible canister sized to receive a surgical device in the canister lumen;
(b) a top cap coupled to a proximal end of the flexible canister, the top cap comprising a top cap housing and a top cap lumen defined in the top cap housing, wherein the at least one top cap lumen is configured to receive a portion of the surgical device slidably disposed therethrough; and
(c) an incision port coupled to a distal end of the flexible canister, the incision port comprising at least one downwardly extending ledge, the at least one downwardly extending ledge configured to facilitate coupling an upper ring portion of a fluidic sealing component to the incision port, the fluidic sealing component configured to provide a fluidic seal at the incision port.

2. The surgical insertion device of claim 1, wherein the at least one downwardly extending ledge comprises an inward shelf portion configured to support the upper ring portion of the fluidic sealing component seated thereon.

3. The surgical insertion device of claim 2, comprising at least two downwardly extending ledges and inward shelf portions disposed about a lumen of the incision port.

4. The surgical insertion device of claim 3, comprising three downwardly extending ledges and inward shelf portions spaced evenly about the lumen of the incision port and configured to receive the upper ring portion of the fluidic sealing component upon flexible positioning of the upper ring portion within the downwardly extending ledges and inward shelf portions.

5. The surgical insertion device of claim 3, further comprising a pivotable ledge portion extending from a lower portion of the incision port, wherein the pivotable ledge portion is configured to pivot between a first insertion position for facilitating positioning of the upper ring portion of the fluidic sealing component within the lower portion of the incision port, and a second engagement position for securing the upper ring portion of the fluidic sealing component to the lower portion of the incision port.

6. The surgical insertion device of claim 5, wherein the pivotable ledge portion is spring-biased towards the second engagement position.

7. The surgical insertion device of claim 1, wherein the at least one downwardly extending ledge comprises a plurality of circumferentially spaced tabs, the circumferentially spaced tabs configured to support the upper ring portion of the fluidic sealing component flexibly coupled to an outer portion of the circumferentially spaced tabs.

8. The surgical insertion device of claim 7, wherein one or more of the circumferentially spaced tabs is configured to flex radially inward to enable positioning of the upper ring portion of the fluidic sealing component around the outer portion of the circumferentially spaced tabs.

9. The surgical insertion device of claim 8, wherein the incision port further comprises at least one removal tabs extending outwardly from the incision port, the at least one removal tab configured to enable removal of the fluidic sealing component from the incision port upon actuation of the removal tab.

10. The surgical insertion device of claim 9, wherein actuation of the removal tab comprises outward or downward movement of the removal tab relative to the incision port, and wherein actuation of the removal tab pulls the upper ring portion of the fluidic sealing component away from the circumferentially spaced tabs.

11. The surgical insertion device of claim 1 wherein the fluidic sealing component is a sealable sleeve device or a wound retractor seal device.

12. A surgical insertion device comprising:
(a) a flexible canister defining a canister lumen, the flexible canister sized to receive a surgical device in the canister lumen;
(b) a top cap coupled to a proximal end of the flexible canister, the top cap comprising a top cap housing and a top cap lumen defined in the top cap housing, wherein the at least one top cap lumen is configured to receive a portion of the surgical device slidably disposed therethrough; and
(c) an incision port coupled to a distal end of the flexible canister, the incision port comprising an iris sealing mechanism disposed therein, the iris sealing mechanism configured to selectively open and close access through a lumen of the incision port upon relative rotation of an upper iris component and a lower iris component.

13. The surgical insertion device of claim 12, wherein the incision port further comprises a ratchet mechanism associated with the iris sealing mechanism, the ratchet mechanism configured to permit relative rotation of the upper iris component and the lower iris component in a first direction towards closure of the iris sealing mechanism.

14. The surgical insertion device of claim 13, wherein the incision port further comprises a release mechanism associated with the iris sealing mechanism, the release mechanism configured to disable the ratchet mechanism upon actuation to permit relative rotation of the upper iris component and the lower iris component in a second direction towards opening of the iris sealing mechanism.

15. The surgical insertion device of claim 12, wherein the incision port further comprises an insufflation port configured to deliver a supply of gas to the lumen of the incision port.

16. The surgical insertion device of claim 15, wherein the insufflation port is configured to deliver gas to the lumen of the incision port below the iris sealing mechanism.

17. The surgical insertion device of claim 15, wherein the insufflation port comprises a valve to selectively permit or restrict flow of gas to the lumen of the incision port.

18. The surgical insertion device of claim 12, wherein the top cap further comprises a top cap seal portion disposed across an upper portion of the top cap lumen, the top cap seal portion having a top cap opening formed therein, the top cap seal portion having a wavy cross-sectional shape to provide flexibility.

19. The surgical insertion device of claim 18, wherein the top cap seal portion is formed of molded silicone having a durometer value in a range of from 00 to 50.

20. The surgical insertion device of claim 18, wherein the top cap seal portion is formed of molded silicone and has a Parylene-C coating.

21. An introducer system for a surgical insertion device, the introducer system comprising:
a pair of downwardly extending introducer legs; and
an operable mechanism to hold the introducer legs apart following insertion of the introducer legs into a top cap opening formed in a top cap seal portion of a surgical insertion device,
wherein the downwardly extending introducer legs are configured to be removed from the top cap opening following positioning of a surgical instrument therethrough.

22. The introducer system of claim 21, wherein the pair of downwardly extending introducer legs are flexibly coupled to one another at an upper portion thereof and biased to move apart upon placement within the top cap opening of the top cap seal portion.

23. The introducer system of claim 21, wherein the pair of downwardly extending introducer legs further comprise tabs configured to seat within a pair of corresponding slots formed in an outer portion of the top cap of the surgical insertion device.

24. The introducer system of claim 21, wherein the pair of downwardly extending introducer legs are rotatably coupled together about a pivot joint, each of the introducer legs having a corresponding handle portion coupled thereto, the handle portions configured to move the introducer legs apart upon movement of the handle portions together following placement of the introducer legs within the top cap opening of the top cap seal portion.

25-30. (canceled)
